(19) United States
(12) Patent Application Publication
(10) Pub. No.: US 2024/0124387 A1
(43) Pub. Date: Apr. 18, 2024

(54) ACID RECOVERY

(71) Applicant: GREENCOVERY B.V., Wageningen (NL)

(72) Inventors: Carlos Iván CABRERA-RODRÍGUEZ, Wageningen (NL); Francisco CAPARRÓS SALVADOR, Wageningen (NL); Joana Patricia Correia LEIRÓS, Wageningen (NL)

(73) Assignee: GREENCOVERY B.V., Wageningen (NL)

(21) Appl. No.: 18/264,008

(22) PCT Filed: Feb. 4, 2022
(86) PCT No.: PCT/NL2022/050056

§ 371 Date: Aug. 2, 2023

(30) Foreign Application Priority Data
Feb. 5, 2021 (NL) 2027501

(51) Int. Cl. C07C 227/40; B01D 15/36; B01D 15/42

(52) U.S. Cl. CPC C07C 227/40; B01D 15/363; B01D 15/426

(57) ABSTRACT

The invention provides a method for the extraction of an organic acid (10) from a mixture (110), the method comprising: an extraction stage comprising providing the mixture (110) to a sorbent (150); an elution stage comprising passing an eluent along the sorbent (120) to provide an eluate (140), wherein the eluent (130) comprises a solvent selected from the group consisting of acetone and alcohols, and wherein the eluent (130) comprises at least 0.1 M of OH—, and wherein the eluent (130) comprises at least 0.1 M of an cation selected from the group comprising Na⁺, K⁺, Ca²⁺, Mg²⁺ and NH⁴⁺; a precipitation stage comprising providing CO₂ (150) to the eluate (140), wherein the CO₂ (150) and the cation form a carbonate, wherein the carbonate precipitates; and a separation stage comprising separating the organic acid (10) from the eluate (140).

### FIELD OF THE INVENTION

The invention relates to a method for the extraction of an acid from a mixture. The invention further relates to a system for recovery of an acid from a mixture. The invention further relates to a computer program product. The invention further relates to a data carrier.

### BACKGROUND OF THE INVENTION

Methods for acid recovery are known in the art. For instance, Rebecchi et al., “Volatile fatty acids recovery from the effluent of an acidogenic digestion process fed with grape pomace by adsorption on ion exchange resins”, Chemical Engineering Journal, Volume 306, 2016, describe a volatile fatty acid separation process from an actual effluent of grape pomace acidogenic anaerobic digestion by ion exchange resins. It described batch ion exchange and desorption tests with acetic acid, volatile fatty acid synthetic mixtures and an actual digestate.

EP3241820 describes a method for recovering a carboxylate, using an anion exchange resin capturing the carboxylate, from an aqueous solution comprising said carboxylate, batch wise or continuously, wherein use of raw material is reduced and if possible re-used.

### SUMMARY OF THE INVENTION

Acids of interest and their salts, such as organic acids, or especially amino acids, may often be present in mixtures with other compounds, complicating the further use of the acids. For instance, an acid may be present in a waste stream, a side stream, or in a mixture resulting from a fermentation process.

Prior art processes for the separation of an acid from a mixture may be complicated, may lack specificity for a particular acid, may be difficult to adapt to different acids, may be costly in terms of reagents and in terms of separation of the acid from an eluent, and may generate large amounts of (undesirable) by-products.

Hence, it is an aspect of the invention to provide an alternative method for acid recovery, which preferably further at least partly obviates one or more of above-described drawbacks. The present invention may have as object to overcome or ameliorate at least one of the disadvantages of the prior art, or to provide a useful alternative.

Hence, in a first aspect, the invention provides a method for the extraction of an organic acid from a mixture (comprising such organic acid). The method may comprise one or more of an extraction stage, an elution stage, and a precipitation stage. In embodiments, the extraction stage may comprise providing the mixture to a sorbent, especially wherein (at least part of) the organic acid associates with the sorbent. In further embodiments, the elution stage may comprise passing an eluent along the sorbent, especially wherein the organic acid dissolves in the eluent, to provide an (acid-containing) eluate. The eluent may especially comprise a solvent selected from the group consisting of acetone and alcohols. Further, the eluent may comprise at least 0.1 M of OH⁻, and the eluent may especially comprise at least 0.1 M of an ion, especially a cation, selected from the group comprising Na⁺, K⁺, Ca²⁺, Mg²⁺ and NH₄⁺. In embodiments, the eluent may regenerate the sorbent during the elution stage, i.e., the elution stage may comprise regenerating the sorbent (by passing the eluent along the sorbent). In further embodiments, the precipitation stage may comprise providing CO₂ to the eluate, especially wherein the CO₂ and the ion form a carbonate, and especially wherein the carbonate precipitates. In further embodiments, the separation stage may comprise separating the organic acid from the eluate, especially via one or more of evaporation, distillation, crystallization, filtration, esterification (of the organic acid), and acidification.

The invention may provide the benefit that organic acids may be recovered from a mixture efficiently while forming little waste product. In particular, organic acids with a low pKa and/or a low solubility in alcohol may be recovered efficiently with the method of the invention. Further, the method of the invention may facilitate recovering a specific acid, especially via the choice of sorbent. The eluent may enable recovering acids with a low pKa or poor solubility in alcohol, especially while also regenerating the sorbent. Further, the CO₂ exposure may enable efficiently removing the ion—and the OH⁻—from the eluate.

In particular, the eluent may comprise a salt, such as NaOH, which provides the Na⁺ ion as well as OH⁻. The presence of the OH⁻ may substantially improve the solubility of the organic acid in the eluent; for instance, an organic acids may dissolve poorly in acetone/alcohols/water or mixtures of them, but the solubility may increase drastically in the presence of OH⁻. Hence, the base, especially the ion and the OH⁻, may facilitate recovering the organic acid from the sorbent to provide an acid-containing eluate, i.e., the organic acid may (better) dissolve in the eluent due to the OH⁻. Subsequently, the addition of CO₂ to the eluate may result in the ion and (at least part of) the CO₂ forming a carbonate, which may precipitate, effectively removing the ion from the eluate. Similarly, the addition of the CO₂ to the eluate may acidify the eluate, resulting in a reduction of OH⁻, which may reduce the solubility of the acid in the eluate. Further, the acidification of the eluate may result in protonation of the acid, which may reduce the solubility of the acid in the eluate.

In particular, following the CO₂ addition, the eluate may largely consist of the solvent, the organic acid, and optionally water. The organic acid may subsequently be separated from the eluate in a separation stage, for instance via one or more of evaporation, distillation, crystallization, filtration, esterification (of the organic acid), and acidification. In embodiments, the separation stage may comprise adding more CO₂ to separate the acid by adjusting the pH of the eluate and/or by protonating the acid (also see below).

Hence, in specific embodiments, the invention may provide a method for the extraction of an organic acid from a mixture, the method comprising: an extraction stage comprising providing the mixture to a sorbent; an elution stage comprising passing an eluent along the sorbent to provide an eluate, wherein the eluent comprises a solvent selected from the group consisting of acetone and alcohols, and wherein the eluent comprises at least 0.1 M of OH⁻, and wherein the eluent comprises at least 0.1 M of a cation selected from the group comprising Na⁺, K⁺, Ca²⁺, Mg²⁺ and NH₄⁺; a precipitation stage comprising providing CO₂ to the eluate, wherein (at least part of) the CO₂ and the cation form a carbonate, wherein the carbonate precipitates; and a separation stage comprising separating the organic acid from the eluate.

Hence, the invention may provide a method for the extraction of an acid, especially an organic acid, from a mixture (comprising such acid).

The term “acid” may herein especially refer to a compound comprising at least one acid group, especially a compound comprising at least one carboxyl group. In embodiments, the acid may comprise an organic acid, i.e., an organic compound comprising at least one acid group, such as a carboxylic acid. In further embodiments, the (organic) acid may comprise an amino acid; especially, the (organic) acid may be an amino acid. The term “amino acid” may herein refer to an organic compound comprising an amino and a carboxyl functional group, along with a side chain. In specific embodiments, the amino acid may comprise a proteinogenic amino acid.

In embodiments, the acid may comprise an organic acid, especially an amino acid.

In further embodiments, the acid may comprise an organic acid, especially an organic acid selected from the group comprising lactate, caproate, acetate, propionate, butyrate, isobutyrate, valerate, isovalerate, heptanoate, octanoate and galacturonate. In particular, the acid may in embodiments comprise an organic acid selected from the group comprising lactate, caproate, and acetate

In further embodiments, the acid may comprise an amino acid, especially a proteinogenic amino acid, such as an amino acid selected from the group comprising histidine, isoleucine, leucine, methionine, phenylalanine, threonine, tryptophane, valine, and lysine, galacturonic acid, aspartic acid, glycine, and/or especially a non-preoteinogenic amino acid, such as an amino acid selected from the group comprising betaine, 4-amino benzoic acid and gamma-amino butyric acid

The term “mixture” may herein especially refer to a mixture of two or more compounds, such as at least the acid and one or more additional compounds, such as on or more second solvents. In embodiments, the mixture may especially be a liquid mixture.

In embodiments, the method may comprise one or more of an extraction stage, an elution stage, a precipitation stage, and a separation stage.

The extraction stage may comprise providing the (liquid) mixture to a sorbent, especially wherein (at least part of) the acid may sorb to the sorbent. Especially, the extraction stage may comprise providing the (liquid) mixture to an adsorbent, especially wherein the acid may adsorb to the adsorbent.

The phrase “providing the mixture to a sorbent” may herein also in embodiments refer to bringing the sorbent into contact with the mixture or bringing the mixture into contact with the sorbent or mixing the sorbent and the mixture together. In particular, the extraction stage may comprise contacting the (liquid) mixture and a sorbent, especially wherein (at least part of the total number of) the acid (molecules) may sorb to the sorbent. In practice, generally, the sorbent may be arranged in a container, such as in a column, and the mixture may be provided to the container.

In embodiments, the sorbent may especially comprise a chromatographic resin, such as an ion exchange resin. The term “ion exchange resin” may herein refer to a resin or polymer that may act as a medium for ion exchange.

In further embodiments, the sorbent may comprise an anion exchange resin, especially a weak anion exchange resin, or especially a strong anion exchange resin.

The terms “weak anion exchange resin” and “strong anion exchange resin” may be known in the art. In particular, the term “weak anion exchange resin” may herein refer to a weak exchanger that is only ionized over a limited pH range, especially to an exchanger that can take up or lose protons depending on changes on the buffer pH, whereas a strong anion exchange resin may herein refer to an exchanger that shows (essentially) no variation in capacity with pH changes, especially to an exchanger that remains fully charged over a broad pH range.

The choice of sorbent may influence the specificity for the acid (with respect to other compounds in the mixture). In particular, the mixture may comprise a plurality of (different) acids, and the sorbent may be selected such that a proper subset of the plurality of acids sorb to the sorbent. In particular, in embodiments, the sorbent may be selected from the group comprising polystyrene and acrylate ion exchange resins, especially from the group comprising polystyrene and acrylate ion exchange resins with a quaternary, tertiary and/or secondary ammonium group, or especially from the group comprising polystyrene and acrylate ion exchange resins with a carboxyl groups.

Hence, during the extraction stage, the acid may associate with the sorbent.

The elution stage may comprise passing an eluent along, especially through, the sorbent to provide an (acid-containing) eluate, i.e., the acid may dissolve in the eluent as the eluent passes along the sorbent.

Hence, the eluent may be selected to be suitable for dissolving the acid in the eluent. In particular, the acid may have a solubility of at least 1 g/L in the eluent, such as at least 3 g/L in the eluent, especially at least 5 g/L in the eluent, such as at least 6 g/L in the eluent. In further embodiments, the acid may have a solubility of at least 10 g/L in the eluent. In particular, the eluent may comprise an (organic) solvent selected from the group consisting of acetone and alcohols, i.e., from the group consisting of acetone and of organic compounds comprising at least one hydroxyl functional group bound to a saturated carbon atom. In particular, the eluent may comprise a solvent selected from the group consisting of acetone and compounds having the formula CₙHₙ₊₁OH, especially from the group consisting of acetone, methanol and ethanol. In further embodiments, the solvent may comprise ethylene glycol. The term “solvent” may herein also refer to a plurality of solvents, such as two or more (organic) solvents selected from the group consisting of acetone and alcohols.

In further embodiments, the eluent may comprise acetone as (organic) solvent.

In further embodiments, the eluent may comprise an (organic) solvent selected from the group consisting of alcohols, especially from the group consisting of water soluble alcohols, and/or especially from the group consisting of C1-C6 alcohols, such as from the group consisting of C1-C5 alcohols, especially from the group consisting of C1-C4 alcohols. In further embodiments, the alcohols may be primary alcohols, secondary alcohols or tertiary alcohols, especially primary alcohols, or especially secondary alcohols, or especially tertiary alcohols.

In further embodiments, the group consisting of alcohols may especially be a group consisting of (water soluble) diols, such as the group consisting of C1-C6 diols, especially the group consisting of C1-C5 diols, such as the group consisting of C1-C4 diols.

In particular, in embodiments, the eluent may comprise at least 30 wt. % of the (organic) solvent (relative to total weight of the eluent), such as at least 40 wt. %, especially at least 50 wt. %, such as at least 60 wt. %. In further embodiments, the eluent may comprise at least 70 wt. % of the (organic) solvent, such as at least 80 wt. %. In further embodiments, the eluent may comprise at least (about) 90 wt. % of the (organic) solvent, such as at least 93 wt. %, especially at least 94 wt. %, including 100%.

In further embodiments, the eluent may comprise at most 99 wt. % of the (organic) solvent (relative to total weight of the eluent), such as at most 95 wt. %, especially at most 90 wt. %, such as at most 80 wt. %.

In particular, the solvent may be selected to be a solvent with a relatively low solubility for the acid, as this may facilitate separating the acid from the solvent at a later stage. Hence, the acid may have a solubility of at most 3 g/L in the solvent, especially at most 1 g/L in the solvent, such as at most 0.8 g/L in the solvent. In further embodiments, the acid may have a solubility of at most 0.5 g/L in the solvent, especially at most 0.3 g/L in the solvent, such as at most 0.1 g/L in the solvent. It will be clear to the person skilled in the art, that this solubility refers to the solvent as such, i.e., essentially pure solvent, and not to the eluent as applied in the elution stage.

In particular, in embodiments wherein the acid is separated from the solvent via precipitation, it may be beneficial for the acid to have a poor solubility in the solvent as such relative to the solubility of the acid in the eluent. Hence, in embodiments, the solubility of the acid in the eluent may be at least three times the solubility of the acid in the solvent, especially at least five times, such as at least ten times.

In further embodiments, the eluent may further comprise water. In particular, the eluent may comprise at least 1 wt. % of water (relative to total weight of the eluent), such as at least 2 wt. %, especially at least 3 wt. %, such as at least 5 wt. %. In further embodiments, the eluent may comprise at least 7 wt. % of water, such as at least 9 wt. %, especially at least 10 wt. %. In further embodiments, the eluent may comprise at least (about) 12 wt. % of water, such as at least 15 wt. %, especially at least 20 wt. %.

In further embodiments, the eluent may comprise at most 50 wt. % of water (relative to total weight of the eluent), such as at most 40 wt. %, especially at most 25 wt. %, such as at most 20 wt. %. In further embodiments, the eluent may comprise at most 12 wt. % of water, such as at most 10 wt. %, especially at most 8 wt. %.

The phrase “the eluent may comprise at least 30 wt. % of X”, and similar phrases, herein refers to at least 30 wt. % of the eluent consisting X, i.e., X makes up at least 30 wt. % of the eluent.

In particular, the solvent together with the water (at their respective weight percentages) may be selected to provide a relatively low solubility for the acid, as this may facilitate separating the acid from the solvent at a later stage. Hence, the acid (or a salt comprising the acid) may have a solubility of at most 5 g/L in the solvent/water mixture, such as at most 3 g/L in the solvent/water mixture, especially at most 1 g/L in the solvent/water mixture, such as at most 0.8 g/L in the solvent/water mixture. In further embodiments, the acid (or especially a salt comprising the acid) may have a solubility of at most 0.5 g/L in the solvent/water mixture, especially at most 0.3 g/L in the solvent/water mixture, such as at most 0.1 g/L in the solvent/water mixture. It will be clear to the person skilled in the art that this solubility refers to the solvent and water mixture as such, i.e., not to the eluent as applied in the elution stage.

In particular, in embodiments wherein the acid is separated from the solvent/water mixture via precipitation, it may be beneficial for the acid to have a poor solubility in the solvent/water mixture as such relative to the solubility of the acid in the eluent. Hence, in embodiments, the solubility of the acid in the eluent may be at least three times the solubility of the acid in the solvent/water mixture, especially at least five times, such as at least ten times.

In particular, in embodiments, the eluent may further comprise at least 0.005 M of OH⁻, especially at least 0.01 M of OH⁻, such as at least 0.05 M of OH⁻, especially at least 0.1 M of OH⁻, such as at least 0.2 M of OH⁻. In further embodiments, the eluent may comprise at most 3 M of OH⁻, such as at most 1.5 M, especially at most 1 M, such as at most 0.5 M of OH⁻, especially at most 0.2 M of OH⁻. In further embodiments, the eluent may comprise 0.01-1.5 M of OH⁻.

The presence of OH⁻ in the eluent may increase the solubility of the acid in the eluent. Hence, although the acid may not dissolve easily in the solvent as such, it may dissolve well in the eluent due to the OH⁻, especially due to the elevated pH. In embodiments, the eluent may have a pH≥5.5, especially ≥6, such as ≥6.5, especially ≥7. Such pH values may be suitable for a large range of organic acids. Amino acids may generally have relatively large pKa values. Hence, for amino acids an eluent with a large pH may be selected. In particular, in further embodiments, the eluent may have a pH≥8.5, such as ≥9, especially ≥9.5. In further embodiments, the eluent may have a pH≤14, especially ≤13, such as ≤12.

In further embodiments, the acid may have a pKa P1, wherein the eluent may have a pH≥P1+0.5, especially ≥P1+1, such as ≥P1+1.5.

The term pH with respect to the eluent may herein especially refer to 14+log 10([OH—]), wherein [OH⁻] refers to the concentration of [OH⁻] in the eluent.

The pH of the eluent may especially be determined using a pH strip.

The OH⁻ may especially be provided to the eluent as a salt.

Hence, in embodiments, the eluent may further comprise at least 0.01 M of an ion, especially a cation, such as at least 0.05 M, especially at least 0.1 M, such as at least 0.2 M. In further embodiments, the eluent may comprise at most 3 M of the cation, such as at most 1.5 M, especially at most 1 M, such as at most 0.5 M, especially at most 0.2 M.

The cation may especially be selected to be suitable for forming a carbonate when exposed to CO₂. Hence, the cation may together with CO₂ form a carbonate (in the carbonate precipitation stage; see below). Specifically, the CO₂ may form H₂CO₃, i.e., carbonic acid, (together with water in the eluate), and the cation may react with HCO₃⁻ or with CO₃²⁻ to form a carbonate.

For example, in embodiments, the cation may especially be selected from the group comprising Li, Be, Al, Cr, Mn, Co, Ni, Cu, Zn, Rb, Sr, Pd, Ag, Cd, Cs, Ba, Na, K, Ca, Mg and NH₄, especially from the group comprising Na, K, Ca, Mg and NH₄. In embodiments, the cation may especially be selected from the group comprising Na⁺, K⁺, Ca²⁺, Mg²⁺ and NH₄⁺.

For instance, the method may thus comprise an eluent preparation step, wherein the eluent preparation step comprises adding a salt to a solvent, wherein the salt comprises the cation and OH⁻, such as a salt selected from the group comprising NaOH, KOH, Ca(OH)₂, Mg(OH)₂ and NH₄OH.

Hence, in embodiments, the eluent may comprise at least 0.1 M of a salt, such as at least 0.05 M, especially at least 0.1 M, such as at least 0.2 M. In further embodiments, the eluent may comprise at most 3 M of the salt, such as at most 1.5 M, especially at most 1 M, such as at most 0.5 M, especially at most 0.2 M. The salt may especially be selected from the group comprising inorganic hydroxides, more especially from the group comprising sodium hydroxide, potassium hydroxide, calcium hydroxide, magnesium hydroxide and ammonium hydroxide.

In embodiments, the eluent may regenerate the sorbent during the elution stage. In particular, in embodiments wherein the sorbent comprises an anion exchange resin, the OH⁻ from the eluent may exchange with the acid from the resin, thereby regenerating the resin.

In embodiments, the method may comprise a (carbonate) precipitation stage. The carbonate precipitation stage may comprise providing CO₂ to the (acid-containing) eluate. Thereby, the CO₂ and the cation may form a carbonate. Carbonates may generally have a (relatively) poor solubility. Hence, the formed carbonate may precipitate during the carbonate precipitation stage.

In embodiments, the carbonate precipitation stage may comprise providing CO₂ to the (acid-containing) eluate such that at least 30% of the cation precipitates (as a carbonate), especially at least 50%, such as at least 70%. In further embodiments, the carbonate precipitation stage may comprise providing CO₂ to the (acid-containing) eluate such that at least 80% of the cation precipitates (as a carbonate), especially at least 90%, such as at least 95%.

In embodiments, the eluent may comprise n1 M of a cation, such as sodium, and the carbonate precipitation stage may comprise providing n2 M of CO₂, wherein n2 is at least 0.1*n1, such as at least 0.2*n1, especially at least 0.25*n1. In particular, even adding a relatively small amount of CO₂ relative to the cation may already provide a desired effect on the solubility of the acid. In further embodiments, n2 may be at least 0.5*n1, such as at least 1*n1. In further embodiments, n2 may be at least 1.5*n1, such as at least 2*n1, especially at least 3*n1. In further embodiments, n2 may be at most 1000*n1, such as at most 500*n1, especially at most 200*n1. In further embodiments, n2 may be at most 100*n1, such as at most 50*n1, especially at most 10*n1. In further embodiments, n2 may be at most 5*n1, such as at most n1, especially at most 0.5*n1.

The term “carbonate” may herein especially refer to a compound comprising the carbonate anion (CO₃²⁻), especially to a salt, ester, or mineral comprising the carbonate anion. For instance, the term “carbonate” may herein refer to any one of HCO₃⁻, H₂CO₃, HNaCO₃, Na₂CO₃, CaCO₃, and MgCO₃.

For instance, in specific embodiments, the cation may comprise sodium, and the carbonate may comprise sodium carbonate.

In embodiments, the carbonate precipitation stage may comprise providing the CO₂ via sparging.

Further, the addition of CO₂ to the eluate may result in the formation of carbonic acid (H₂CO₃), which may behave as a dibasic acid, forming an equilibrium with HCO₃⁻ and CO₃²⁻, thereby providing some acidification of the eluate, especially a reduction of the amount of OH⁻, and especially a corresponding reduction of the pH.

As indicated above, the acid may have a poor solubility in the solvent (optionally together with the water), and may have an improved solubility in the eluent due to the presence of the OH⁻. Then, as the addition of CO₂ reduces the amount of OH⁻, also the solubility of the acid may decrease. Simultaneously, the addition of the CO₂ may remove other compounds from the eluate, especially remove the cation from the eluate. Thereby, the precipitation stage may provide an (acid-containing) eluate comprising the solvent and the acid, and optionally the water, from which the acid may be relatively easily separated.

Hence, in embodiments, the method may further comprise a separation stage. The separation stage may comprise separating the acid from the (acid-containing) eluate, especially via one or more separation steps selected from the group comprising evaporation, distillation, crystallization, filtration, esterification (of the organic acid), and acidification. The person skilled in the art will select an appropriate separation step in view of the acid and the solvent.

Hence, the method of the invention may facilitate selectively separating an acid from a mixture using a sorbent, providing an eluent to the sorbent in which the acid dissolves (relatively) well to provide an acid-containing eluate, altering the solubility of the acid in the eluate by acidification of the eluate while simultaneously removing other compounds from the eluate, to facilitate an easy separation of the acid from the eluate in the separation stage. In particular, the method of the invention may result in relatively little by-product (or “waste-products”).

In embodiments, the acid may have a pKa selected from the range of 1.5-11, especially from the range of 2-10, such as from the range of 3-8, especially from the range of 4-6.

In particular, recovering organic acids with a low pKa may be challenging with prior art methods, especially for prior art methods relying on protonation of the acid. The method of the invention may be particularly suitable for separating organic acids with low pKa values. Hence, in embodiments, the organic acid may have a pKa≤5.0, such as ≤4.5. In further embodiments, the organic acid may have a pKa≤4.0, especially ≤3.5, such as ≤3.0. In further embodiments, the organic acid may have a pKa≤2. In further embodiments, the acid may have a pKa≥1, especially ≥2, such as ≥3.

In embodiments, the sorbent may be selected from the group comprising a weak anion exchange resin, a strong anion exchange resin, and an adsorbent, especially from the group comprising a weak anion exchange resin, and a strong anion exchange resin.

The carbonate precipitation stage may comprise adding the CO₂ at a predefined temperature. The predefined temperature may be selected in view of several competing criteria. In particular, the solubility of the CO₂ in the eluate may advantageously be higher at a lower temperature. However, the solubility of the acid may be lower at a lower temperature, and cooling of the CO₂ may be energy-intensive.

Hence, in embodiments, the carbonate precipitation stage may comprise providing the CO₂ at a temperature selected from the range of 0-50° C., especially from the range of 5-40° C., such as from the range of 10-25° C. In particular, the temperature of the CO₂ may affect the solubility of the CO₂ in the eluate. In particular, in embodiments, the carbonate precipitation stage may comprise providing CO₂ having a temperature selected from the range of 0-50° C., especially from the range of 5-40° C., such as from the range of 10-25° C.

In embodiments, the carbonate precipitation stage may comprise separating the (acid-containing) eluate and the precipitated carbonate. For instance, the method may comprise passing the eluate through a sieve, or may comprise (continuously) removing precipitate (from a vessel).

The solubility of the acid in the eluate may further decrease if the pH is lowered further. Hence, in embodiments, the separation stage may comprise providing CO₂ to the eluate to (gradually) reduce the pH of the eluate to a precipitation point of (a conjugate base of) the acid.

Hence, in embodiments, the method may comprise first adding CO₂ to remove the cation (and the OH⁻) in the precipitation stage, and may subsequently comprise adding additional CO₂ to reduce the pH of the eluate in the separation stage.

The precipitation stage and the separation stage may, in embodiments, be temporally separated, and/or may be executed at different locations. For instance, the precipitation stage may be executed in a first vessel, after which the eluate is provided from the first vessel to a second vessel, especially via a sieve, and the separation stage may be executed in the second vessel.

In further embodiments, the precipitation stage may smoothly transition to the separation stage, especially wherein providing of the CO₂ is not interrupted between the two stages.

Hence, in embodiments, the method may comprise providing CO₂ to reduce the pH of the eluate to a separation point, such as a precipitation point and/or a phase separation point of the acid, especially to a precipitation point, or especially to a phase separation point. The term “precipitation point” may herein especially refer to a pH at which a compound precipitates from a solution. The term “phase separation point” may herein especially refer to a pH at which a phase separation occurs, especially wherein the acid is separated from a remainder of the eluate during the phase separation, i.e., the phase separation may provide an acid fraction which is enriched in acid relative to the eluate.

In particular, an acid may precipitate from a solution at a pH wherein the acid has a neutral charge. Hence, the precipitation point may lie close to an isoelectric point p1 of the acid. Hence, in embodiments, the acid may have an isoelectric point pI, wherein the precipitation point may be selected from the range of pI−1-pI+1, especially from the range of pI−0.5-pI+0.5, such as from the range of pI−0.2-pI+0.2.

In embodiments, the acid may have a dissociation constant pKa, wherein the separation point, especially the precipitation point, or especially the phase separation point, may be selected from the range of <pKa.

In further embodiments, the acid may have a plurality of dissociation constants, such as a lower dissociation constant pKa₁ and an upper dissociation constant pKaᵤ, wherein the precipitation point may be selected from the range of <pKaᵤ, and especially from the range of >pKa₁, or especially from the range of <pka₁.

In further embodiments, the acid may comprise two or more (different) (organic) acid compounds, wherein the separation stage comprises providing CO₂ to the eluate to successively reduce the eluate to the precipitation points of the two or more organic acid compounds to separate the two or more organic acid compounds. Hence, the method, especially the separation stage, may comprise first providing CO₂ to the eluate to reduce the eluate to the precipitation point of a first acid compound of the two or more acid compounds (to separate the first acid compound), and subsequently providing CO₂ to the eluate to reduce the eluate to the precipitation point of a second acid compound of the two or more acid compounds (to separate the second acid compound).

In particular, in embodiments, the first acid compound may have a first isoelectric point pI1, and the second acid compound may have a second isoelectric point pI2, wherein pI2<PI1, and the method, especially the separation stage, may comprise first providing CO₂ to the eluate to reduce the eluate to a pH selected from the range of pI1−0.5-pI1+0.5, and subsequently providing CO₂ to the eluate to reduce the eluate to a pH selected from the range of pI2−0.5-pI2+0.5.

The method of the invention may be particularly suitable to recover an acid from a waste stream, such as a by-product stream, or such as a general (complex) waste stream. Hence, in embodiments, the mixture may be (obtained from) a waste stream.

In a further aspect, the invention may provide a system for recovery of an acid, especially an organic acid, from a (liquid) mixture. The system may comprise one or more of an inlet, a sorbent container, an eluent supply, a separation unit, and a carbon dioxide supply.

The sorbent container may especially be configured to host a sorbent. Hence, especially during operation, the system may comprise a sorbent arranged in the sorbent container.

In embodiments, the eluent supply may be configured to provide an eluent to the sorbent container, especially to the sorbent, especially wherein the eluent passes along, such as through, the sorbent. In further embodiments, the separation unit may be configured to receive an eluate from the sorbent container, especially from the sorbent.

The term “eluent” may herein especially refer to a liquid provided to the sorbent, whereas the term “eluate” may herein especially refer to the liquid after having passed (through) the sorbent. Hence, the eluent supply may be configured to pass an eluent through the sorbent to provide an eluate.

In further embodiments, the system, especially the control system (see below), may have an operational mode. The term “operational mode” may also be indicated as “controlling mode”. The system, or apparatus, or device (see further also below) may execute an action in a “mode” or “operational mode” or “mode of operation”. Likewise, in a method an action, stage, or step may be executed in a “mode” or “operation mode” or “mode of operation”. This does not exclude that the system, or apparatus, or device may also be adapted for providing another operational mode, or a plurality of other operational modes. Likewise, this does not exclude that before executing the mode and/or after executing the mode one or more other modes may be executed. However, in embodiments a control system (see further also below) may be available, that is adapted to provide at least the operational mode. Would other modes be available, the choice of such modes may especially be executed via a user interface, though other options, like executing a mode in dependence of a sensor signal or a (time) scheme, may also be possible. The operational mode may in embodiments also refer to a system, or apparatus, or device, that can only operate in a single operational mode (i.e. “on”, without further tunability).

The operational mode may comprise one or more of an extraction stage, an elution stage, a (carbonate) precipitation stage, and a separation stage.

In embodiments, in the extraction stage, the inlet may (be configured to) provide the mixture to the sorbent container, especially to the sorbent, especially wherein the acid associates with the sorbent.

In further embodiments, in the elution stage, the eluent supply may (be configured to) provide an eluent to the sorbent container, especially to the sorbent, to provide an (acid-containing) eluate to the eluate receiver. The eluent may especially comprise at least 40 wt. % of an (organic) solvent selected from the group consisting of acetone and alcohols. In specific embodiments, the eluent may comprise at least 0.1 M of OH⁻, and/or the eluent may comprise at least 0.1 M of a cation selected from the group comprising Na⁺, K⁺, Ca²⁺, Mg²⁺ and NH₄⁺, especially a monovalent cation, such as a monovalent cation selected from the group comprising Na⁺, K⁺, and NH₄⁺, or especially a bivalent cation, such as a bivalent cation selected from the group comprising Ca²⁺, Mg²⁺.

In further embodiments, in the (carbonate) precipitation stage, the carbon dioxide supply may (be configured to) provide CO₂ to the separation unit. Especially, (at least part of) the CO₂ and the cation may form a carbonate, especially wherein the carbonate precipitates.

In particular, the solubility of the carbonate may depend on the cation. Hence, the cation may be selected in view of the solubility of the corresponding carbonate, and in view of the to be recovered acid.

In the separation stage, the separation unit may (be configured to) separate the acid from the eluate, especially via one or more of evaporation, distillation, crystallization, filtration, esterification, and acidification. Hence, in embodiments, the separation unit may comprise one or more of an evaporation unit, a distillation unit, a crystallization unit, a filtration unit, an esterification unit and an acidification unit.

Hence, in specific embodiments, the invention may provide a system for recovery of an acid, especially an organic acid, from a (liquid) mixture, wherein the system comprises an inlet, a sorbent container, an eluent supply, a separation unit, and a carbon dioxide supply, wherein the sorbent container is configured to host a sorbent, wherein the eluent supply is configured to provide an eluent to the sorbent container, especially to the sorbent, and wherein the separation unit is configured to receive an eluate from the sorbent container, especially from the sorbent, wherein the system comprises an operational mode, wherein: the inlet provides the mixture to the sorbent container, especially to the sorbent; the eluent supply provides an eluent to the sorbent container, especially to the sorbent, to provide an eluate to the eluate receiver, wherein the eluent comprises at least 30 wt. %, such as at least 40 wt. %, especially at least 60 wt. %, of a solvent selected from the group consisting of acetone and alcohols, wherein the eluent comprises at least 0.1 M of OH⁻, and wherein the eluent comprises at least 0.1 M of a cation selected from the group comprising Na⁺, K⁺, Ca²⁺, Mg²⁺ and NH₄⁺; the carbon dioxide supply provides CO₂ to the separation unit, wherein the CO₂ and the ion form a carbonate, wherein the carbonate precipitates; and the separation unit separates the organic acid from the eluate.

In embodiments, the sorbent may be arranged in a sorbent container, especially a column. Hence, the system may comprise a sorbent container comprising the sorbent, especially wherein the inlet is configured to provide the eluent to the sorbent container, and wherein the sorbent container is configured to provide the eluate to the separation unit.

In embodiments, the system may further comprise a control system, especially wherein the control system is configured to control one or more of the inlet, the sorbent container, the eluent supply, the separation unit, and the carbon dioxide supply.

The term “controlling” and similar terms herein may especially refer at least to determining the behavior or supervising the running of an element. Hence, herein “controlling” and similar terms may e.g. refer to imposing behavior to the element (determining the behavior or supervising the running of an element), etc., such as e.g. measuring, displaying, actuating, opening, shifting, changing temperature, etc. Beyond that, the term “controlling” and similar terms may additionally include monitoring. Hence, the term “controlling” and similar terms may include imposing behavior on an element and also imposing behavior on an element and monitoring the element. The controlling of the element can be done with a control system. The control system and the element may thus at least temporarily, or permanently, functionally be coupled. The element may comprise the control system. In embodiments, the control system and the element may not be physically coupled. Control can be done via wired and/or wireless control. The term “control system” may also refer to a plurality of different control systems, which especially are functionally coupled, and of which e.g. one control system may be a control system and one or more others may be slave control systems.

In embodiments, the separation unit may comprise a first separation unit and a second separation unit, especially wherein the first separation unit is configured to receive an eluate from the sorbent. In such embodiments, in the operational mode, especially in the precipitation stage, the carbon dioxide supply may (be configured to) provide CO₂ to the first separation unit, wherein the first separation unit (is configured to) separate the precipitated carbonate and (remaining) eluate, and to provide the (remaining) eluate to the second separation unit. In further embodiments, in the operational mode, especially in the separation stage, the second separation unit may (be configured to) separate the acid from the eluate, especially via one or more of evaporation, distillation, crystallization, filtration, esterification, and acidification. In particular, in further embodiments, in the separation stage, the carbon dioxide supply may (be configured to) provide CO₂ to the second separation unit, especially to (gradually) reduce the pH of the eluate to a precipitation point of the acid, and/or especially to protonate the acid.

In embodiments, the system may be configured to execute the method of the invention. Especially, the control system may be configured to (have the system) execute the method of the invention.

In a further aspect, the invention may provide a computer program product comprising instructions for execution on a control system functionally coupled to a system, wherein the instructions, when executed by the control system, cause the system to carry out the method of the invention. In a further aspect, the invention may provide a data carrier, carrying thereupon program instructions which, when executed by a control system functionally coupled to a system, cause the system to carry out the method of the invention.

In yet a further aspect, the invention (thus) provides a software product, which, when running on a computer is capable of bringing about (one or more embodiments of) the method as described herein.

The term “stage” and similar terms used herein may refer to a (time) period (also “phase”) of a method and/or an operational mode. The different stages may (partially) overlap (in time). For example, the precipitation stage may, in general, be initiated prior to the separation stage, but may partially overlap in time therewith. However, for example, the extraction stage may typically be completed prior to the elution stage. It will be clear to the person skilled in the art how the stages may be beneficially arranged in time.

The embodiments described herein are not limited to a single aspect of the invention. For example, an embodiment describing the method may, for example, further relate to the system, especially to an operational mode of the system, or especially to the control system. Similarly, an embodiment of the system describing an operation of the system may further relate to embodiments of the method. In particular, an embodiment of the method describing an operation (of the system) may indicate that the system may, in embodiments, be configured for and/or be suitable for the operation.

### BRIEF DESCRIPTION OF THE DRAWINGS

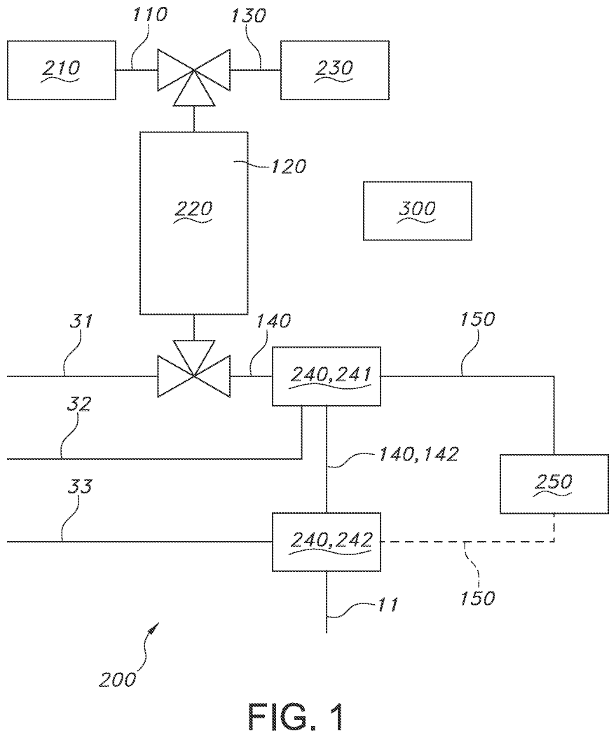

Embodiments of the invention will now be described, by way of example only, with reference to the accompanying schematic drawings in which corresponding reference symbols indicate corresponding parts, and in which: FIG. 1 schematically depicts embodiments of the method and the system of the invention. The schematic drawings are not necessarily on scale.

### DETAILED DESCRIPTION OF THE EMBODIMENTS

FIG. 1 schematically depicts an embodiment of the method of the invention. Specifically, FIG. 1 depicts a method for the extraction of an acid, especially an organic acid 10, from a (liquid) mixture 110. In the depicted embodiment, the method comprises an extraction stage, an elution stage, a (carbonate) precipitation stage and a separation stage.

The extraction stage may comprise providing the mixture 110 to a sorbent 120, whereby the organic acid 10 associates with the sorbent 120. In particular, a remainder of the mixture 110 may leave the sorbent 120 via a first side stream 31.

The elution stage may comprise passing an eluent 130 along the sorbent 120 to provide an (acid-containing) eluate 140, i.e., the organic acid 10 may dissolve in the eluent 130 as the eluent 130 passes along, especially through, the sorbent 120. In embodiments, the eluent 130 may comprise a solvent selected from the group consisting of acetone and alcohols. In further embodiments, the eluent may comprise water. In further embodiments, the eluent 130 may comprise at least 0.1 M of OH⁻. In further embodiments, the eluent may comprise at least 0.1 M of a cation, especially a cation selected from the group comprising Na⁺, K⁺, Ca²⁺, Mg²⁺ and NH₄⁺. Further, as the eluent 130 passes along the sorbent 120, especially wherein the sorbent 120 comprises an anion exchange resin, the eluent 130 may regenerate the sorbent 120.

The (carbonate) precipitation stage may comprise providing CO₂ 150 to the (acid-containing) eluate 140, wherein the CO₂ 150 and the cation form a carbonate, especially wherein the carbonate precipitates. In embodiments, the (precipitated) carbonate and remaining eluate 140,142 may be separated, such as via filtration. In the depicted embodiment, a second side stream 32 schematically represents separation, especially removal, of the (precipitated) carbonate.

The separation stage may comprise separating the organic acid 10 from the (acid-containing) eluate 140, especially via one or more of evaporation, distillation, crystallization, filtration, esterification of the organic acid 10, and acidification. Hence, the method may provide a product stream 11 comprising the organic acid 10, and may provide a third side stream 33 comprising a remainder of the (remaining) eluate 140, 142.

In embodiments, the precipitation stage may comprise providing the CO₂ 150 at a temperature selected from the range of 0-50° C., especially from the range of 5-40° C., such as from the range of 10-25° C. In particular, the temperature of the CO₂ may affect the solubility of the CO₂ in the eluate.

In further embodiments, the separation stage may comprise providing CO₂ 150 to the eluate 140 to (gradually) reduce the pH of the eluate 140 to a precipitation point of the organic acid 10, especially wherein the precipitation point is within 0.2 from the isoelectric point of the organic acid 10.

In further embodiments, the organic acid 10 may comprise two or more organic acid compounds, and the separation stage may comprise providing CO₂ 150 to the eluate 140 to successively reduce the eluate to the precipitation points of the two or more organic acid compounds to separate the two or more organic acid compounds.

FIG. 1 further depicts an embodiment of the system 200 for recovery of an organic acid 10 from a (liquid) mixture 110. The system 200 comprises an inlet 210, a sorbent 120, an eluent supply 230, a separation unit 240, and a carbon dioxide supply 250. In embodiments, the system may comprise a sorbent container 220, especially a column, comprising the sorbent. In further embodiments, the system may comprise a control system 300 configured to control the system, especially one or more of the inlet 210, the sorbent container 220, the eluent supply 230, the separation unit 240, and the carbon dioxide supply 250. The eluent supply 230 may especially be configured to provide an eluent 130 to the sorbent 120, especially wherein the separation unit 240 is configured to receive an eluate 140 from the sorbent 120. In particular, the eluent supply 230 may be configured to pass an eluent 130 along the sorbent 120 to provide an (acid-containing) eluate to the separation unit 240.

In embodiments, the system 200, especially the control system 300 may have an operational mode.

In embodiments, in the operational mode, the inlet 210 may (be configured to) provide the mixture 110, especially a liquid mixture, to the sorbent 120, especially to the sorbent container 220 comprising the sorbent 120. In particular, the mixture 110 may follow a path passing along, especially through, the sorbent 120 and leaving the system 200 via a first side stream 31.

In further embodiments, in the operational mode, the eluent supply 230 may (be configured to) provide an eluent 130 to the sorbent 120, especially to the sorbent container 220 comprising the sorbent 120, to provide an (acid-containing) eluate 140 to the separation unit 240.

In further embodiments, in the operational mode, the carbon dioxide supply 250 may be configured to provide CO₂ 150 to the separation unit 240. In particular, the CO₂ 150 and the cation (see above) may form a carbonate, wherein the carbonate precipitates. In particular, in the operational mode, the carbonate (precipitate) and the remaining eluate may be separated, such as via filtration. In FIG. 1, the separation may be schematically depicted by the carbonate precipitate leaving the system 200 via the second side stream 32.

In further embodiments, the separation unit 240 may be configured to separate the organic acid 10 from the eluate 140, especially via one or more of evaporation, distillation, crystallization, filtration, esterification, and acidification. In particular, in FIG. 1, the third side stream 33 schematically represents a remainder of the eluate, and a product stream 11 comprises the organic acid 10.

In the depicted embodiment, the separation unit 240 comprises a first separation unit 241 and a second separation unit 242, especially wherein the first separation unit 241 is configured to receive an eluate 140 from the sorbent 120, especially from the sorbent container 220 comprising the sorbent 120. In such embodiments, in the operational mode, the carbon dioxide supply 250 may be configured to provide CO₂ 150 to the first separation unit 241, wherein the first separation unit 241 is configured to separate the precipitated carbonate and remaining eluate 140. In particular, the first separation unit 241 may be configured to provide the (remaining) eluate 140 to the second separation unit 242, and especially to remove the (precipitated) carbonate via a second side stream 32. In further embodiments, in the operational mode, the second separation unit 242 may be configured to separate the organic acid 10 from the eluate 140, especially via one or more of evaporation, distillation, crystallization, filtration, esterification, and acidification.

In further embodiments, in the operational mode, the carbon dioxide supply 250 may be configured to provide CO₂ 150 to the second separation unit 242 to (gradually) reduce the pH of the eluate 140 to a precipitation point of the organic acid 10, which may facilitate separation of the organic acid 10 from the eluate 140.

### EXPERIMENTS

Embodiments of the method were experimentally evaluated with different acids, sorbents, and eluents. These experiments are briefly described herein.

Unless specified otherwise, the experiments were performed using the following conditions.
Chemicals—During adsorption and desorption experiments several chemicals were used, such as ethanol absolute (Merck), ethanol 70% vol (VWR) and ethanol 96% vol (VWR), sodium hydroxide (98% SigmaAldrich), potassium hydroxide (98% SigmaAldrich), potassium hydroxide (85% SigmaAldrich), lactic acid (50% Fluka), sodium acetate (>99%), hexanoic acid (>99%), L-alanine (>98% SigmaAldrich), L-leucine (>98% SigmaAldrich), sodium L-glutamate monohydrate (>99% Scharlau). Solutions were prepared by diluting the chemicals with Milli-Q water and, afterwards, all solutions were filtrated using 0.2 μm filters.
Sorbents—Two different resins from SigmaAldrich were tested, Dowex Marathon WBA (weak resin) and Dowex Marathon MSA (strong resin).
Carbon dioxide—CO₂ (>99.7%) was provided by Linde in a pressurized bottle.
Columns packing—A weighed amount of resin (5 g of wet resin) was placed in an adjustable height Omnifit glass column (1 cm of internal diameter×15 cm max. height). Afterwards, the resin was packed with the assistance of a peristaltic pump and gentle shaking.
Equipment—Adsorption and desorption experiments were performed in an AKTA Purifier. The system has an online measurement of conductivity and UV absorbance. The data processing and control software is UNICORN 5.0. The amino acids adsorption+desorption was analyzed with UV absorbance at 210 nm and for organic acids at 260 nm.
Analysis—The (organic) acid content of the different samples was analyzed in a Dionex HPLC, equipped with a UV/Visible Detector. Separation occurred on a Bio-Rad Aminex HPX-87H column. The amino acids content of the different samples was analyzed in UPLC following the protocol described by Meussen et al., “A Fast and Accurate UPLC Method for Analysis of Proteinogenic Amino Acids”, Food Analytical Methods, 2014, which is hereby herein incorporated by reference.
Methods—Adsorption was run until the outgoing concentration of the acid was identical to the feed concentration of the acid, i.e., until the resin was saturated with the acid. After adsorption, the column was washed with Milli-Q water. Desorption was performed using an eluent comprising ethanol and optionally water (varying % of water), comprising a cation, especially sodium or potassium, and OH⁻. After desorption, the column was again washed with Milli-Q water and later on regenerated with a solution of NaOH in water.

### Experiment 1—Adsorption and Desorption of Organic Acids Without an Amino Group

Adsorption and desorption experiments were carried out with a variety of resins, eluents and organic acids as summarized in table 1:

| | | Water | | | |
|---|---|---|---|---|---|
| Experiment | Resin | wt. % | Alcohol | Base | Organic acid |
| Exp OA1 | WBA | 10 | Ethanol | KOH 1M | Lactate |
| Exp OA2 | WBA | 10 | Ethanol | KOH 1M | Caproate |
| Exp OA3 | MSA | 10 | Methanol | KOH 0.85M | Acetate |

The experiments will be further described based on several representative embodiments.

### Example 1. Adsorption and Desorption of Lactate and Caproate in WBA and Precipitation with CO₂

Instead of the equipment mentioned above, for this example the adsorption and desorption experiments were performed in a Thermo Scientific Dionex Ultimate 3000 system. This system has an online measurement of conductivity, pH and UV absorbance and this data acquired was processed using chromatography data system software (Chromeleon™ 6.8 by Thermo Scientific).

Two different adsorption and desorption experiments were performed in a column packed with 6.6 mL of a weak anion exchange resin, one was performed with a solution containing 20 g/L of lactic acid and the other one with a solution with 20 g/L of caproic acid. The total volume of solution passed through the column during adsorption was 30 mL, which corresponds to a total lactic acid/caproic acid mass of 0.6 g fed to the column. The total outlet from adsorption and desorption was collected and afterwards analyzed in an HPLC to quantify the amount of lactate and caproate present in the fractions. A total of 48.1 mg lactate was adsorbed per mL of the resin bed, whereas a total of 38.3 mg of caproate was adsorbed per mL of the resin bed.

After washing the column with miliQ water until conductivity was stable, an eluent was passed along the sorbent to provide an acid-containing eluate. The eluent comprised a solvent consisting of 90 wt. % ethanol and 10 wt. % water, and the eluent comprised 1M of KOH. A total of 18.5 mg of lactate per mL of resin bed were desorbed; while (essentially) all adsorbed caproate was desorbed.

In the precipitation stage, the desorption samples were sparged with CO₂, and the organic acids were quantified again the precipitation stage. There was (essentially) no reduction of lactate and caproate concentration in each solution.

Hence, example 1 demonstrates that the method of the invention has a high overall recovery rate, particularly with regards to the precipitation stage.

### Example 2. Adsorption and Desorption of Acetate in MSA and Precipitation with CO₂

A different adsorption and desorption experiment was performed in a column packed with 4.1 mL of a strong anion exchange resin with 30 mL a solution containing 12.6 g/L of acetate. A total of 50.7 mg of acetate was adsorbed per mL of resin bed.

After washing the column with miliQ water until conductivity was stable, an eluent was passed along the sorbent to provide an acid-containing eluate. The eluent comprised a solvent comprising 90 wt. % methanol and 10 wt. % water, and the eluent comprised 0.85M of KOH. A total of 43.7 mg of acetate per mL of resin bed was desorbed.

The eluate was separated into four samples of 7.5 mL each. The second sample, initially with 24.6 g/L was subjected to a carbonate precipitation stage, specifically through sparging the sample with CO₂, after which the sample contained 24.3 g/L of acetate. Hence, almost all of the organic acid was retained during the precipitation stage.

### Experiment 2— Adsorption and Desorption of Amino Acids

Adsorption and desorption experiments were carried out with a variety of eluents and amino acids as summarized in table 2:

| | | Water | Ethanol | | |
|---|---|---|---|---|---|
| Experiment | Resin | wt. % | wt. % | Base - conc | Amino acid |
| Exp AA1 | WBA | 6.1 | 93.9 | KOH 0.425M | Glutamate |
| Exp AA2 | | 6.1 | 93.9 | KOH 0.425M | Alanine |
| Exp AA3 | | 6.1 | 93.9 | KOH 0.425M | Leucine |
| Exp AA4 | | 10 | 90 | KOH 0.85M | Glutamate |
| Exp AA5 | | 37.6 | 62.4 | NaOH 1M | Glutamate |

The experiments will be further described based on several representative embodiments.

### Example 3. Adsorption and Desorption of Glutamate (AA1), Alanine (AA2) and Leucine (AA3) in WBA and Precipitation with CO₂

The extraction stage comprised providing a mixture to the sorbent, i.e. to 7.4 mL of WBA, and monitoring the absorbance of the mixture having passed through the sorbent at 215 nm using a UV detector. The absorbance at 215 nm in the UV detector became constant after 120 mL of a solution containing 1.92 g/L of glutamate(−). The same was repeated for a mixture comprising leucine (2.5 g/L) and a mixture comprising alanine (2.5 g/L), for which the absorbance became constant after 90 mL and 60 mL respectively. A total of 16.2 mg of glutamate per mL of resin bed were adsorbed. 16.5 mg of alanine per mL of resin bed were adsorbed and 21.5 mg of leucine per mL of resin bed were adsorbed.

After washing the column with miliQ water until conductivity was stable, a solution of 0.425 M KOH in 93.5 wt. % ethanol was flowed through the loaded column. A total of 7.4 mg of glutamate per mL of resin bed was desorbed, 10.5 mg of alanine per mL of resin bed and 16.2 mg of leucine per mL of resin bed.

The samples with the highest content of amino acid were sparged with CO₂ for the precipitation stage and the separation stage, and the amino acids were quantified again afterwards. The reduction of amino acid concentration in solution (in the eluate) was approximately 68% for glutamic acid, 31% for alanine, and approximately >99% for leucine.

### Example 4. Effect of Water and Caustic Variations in the Desorption and Precipitation of Glutamate in WBA

After adsorption in a weak anion exchange resin, 2 different desorption media (compared with example 3) were evaluated. In example 3, 0.425 M of KOH in 93.5 wt. % ethanol was used. In this example 0.85 M KOH in 90 wt. % ethanol (AA4) and 1 M NaOH in 62.4 wt. % ethanol (AA5) were used.

No significant differences were observed between the different ethanol and caustic concentrations during desorption.

The samples with the highest content of amino acid were sparged with CO₂, and the amino acids were quantified again. The reduction of glutamic acid concentration in solution was approximately 90% for the 1M NaOH in 62.4 wt. % ethanol and approximately 74% for the 0.85 M KOH in 90 wt. % ethanol.

Hence, the amount and/or type of base may affect the recovery of the acid after CO₂ exposure during the precipitation stage.

### Experiment 3— Precipitation Experiments

### Example 5. Precipitation with CO₂ in Solutions Containing Different Concentrations of Amino Acids, Alcohols, Cations, and Water

A variety of eluates, comprising different amino acids, and solvent concentrations were subjected to the precipitation stage as summarized in table 3:

| | | Concentration of amino acid in the liquid | | |
|---|---|---|---|---|
| | | Before | After | |
| | | precipitation | precipitation | Precipitate |
| Solvent | Solute | (g/L) | (g/L) | (g/L) |
| 50 wt. % water | Leucine | 10.2 | 10.2 | 12.6 |
| 50 wt. % ethanol | Methionine | 10.3 | 9.6 | 15.2 |
| 0.5M NaOH | Sodium glutamate | 10.1 | 10.8 | 17.5 |
| | Phenylalanine | 10.3 | 10.3 | 16.9 |
| | Lysine | 10.3 | 10.6 | 25.9 |
| | — | 0.0 | 0.0 | 18.6 |
| 37.6 wt. % water | Leucine | 9.6 | 9.7 | 18.1 |
| 62.4 wt. % ethanol | Methionine | 10.2 | 9.6 | 21.2 |
| 0.5M NaOH | Glutamic acid | 10.1 | 4.8 | 30.5 |
| | Sodium glutamate | 10.1 | 4.3 | 34.1 |
| | Phenylalanine | 10.2 | 9.1 | 20.6 |
| | Lysine | 10.4 | 10.3 | 22.0 |
| | — | 0.0 | 0.0 | 22.0 |
| 20 wt. % water | Leucine | 10.3 | 9.1 | 20.7 |
| 80% wt. % ethanol | Methionine | 10.2 | 4.1 | 27.4 |
| 0.5M NaOH | Glutamic acid | 10.1* | 0.7 | 33.7 |
| | Sodium glutamate | 10.1* | 0.8 | 35.5 |
| | Phenylalanine | 10.3 | 3.6 | 27.4 |
| | Lysine | 10.3 | 3.4 | 32.5 |
| | — | 0.0 | 0.0 | 23.5 |

*Not completely dissolved.

The precipitate in the 50 wt. % ethanol experiments is mainly due to the formation of sodium bicarbonate and/or sodium carbonate, as there is essentially no precipitation of amino acids (difference between after and before precipitation is within measurement error, i.e., it can be attributed to sample analysis and handling errors). That is a simple way of separation of the buffer from the amino acid, as the amino acids stays in solution and the buffer forms a precipitate.

The reduction in the amino acid concentration due to the precipitation stage increases with the concentration of ethanol in all observed cases. In particular, amino acids may generally be less soluble in ethanol than in water at neutral conditions, and may thus precipitate more readily in the presence of more ethanol. Hence, for amino acids, the eluent may especially comprise 30-95 wt. % of a solvent selected from the group of acetone and alcohols, especially 40-90 wt. %, such as 45-60 wt. %.

In further embodiments, the eluent may comprise at least 30 wt. % of a solvent selected from the group of acetone and alcohols, such as at least 40 wt. %, especially at least 50 wt. %. In further embodiments, the eluent may comprise at least 55 wt. % of a solvent selected from the group of acetone and alcohols, such as at least 60 wt. %, especially at least 65 wt. % In further embodiments, the eluent may comprise at most 95 wt. % of a solvent selected from the group of acetone and alcohols, such as at most 90 wt. %, especially at most 85 wt. %. In further embodiments, the eluent may comprise at most 80 wt. % of a solvent selected from the group of acetone and alcohols, such as at most 70 wt. %, especially at most 60 wt. %

Example 5 further demonstrates that different acids, especially amino acids, can be separated by the method of the invention based on differences in solubility depending on the eluent, especially on the solvents in the eluent, when adding CO₂. For instance, with 50 wt. % ethanol, both sodium glutamate and leucine both remained in solution when providing CO₂, while with 62.4% ethanol, sodium glutamate partially precipitated, while leucine remained in solution, and with 80 wt. % ethanol, over 90% of the sodium glutamate precipitated, while less than 10% of the leucine precipitated.

The term “plurality” refers to two or more. Furthermore, the terms “a plurality of” and “a number of” may be used interchangeably.

The terms “substantially” or “essentially” herein, and similar terms, will be understood by the person skilled in the art. The terms “substantially” or “essentially” may also include embodiments with “entirely”, “completely”, “all”, etc. Hence, in embodiments the adjective substantially or essentially may also be removed. Where applicable, the term “substantially” or the term “essentially” may also relate to 90% or higher, such as 95% or higher, especially 99% or higher, even more especially 99.5% or higher, including 100%. Moreover, the terms “about” and “approximately” may also relate to 90% or higher, such as 95% or higher, especially 99% or higher, even more especially 99.5% or higher, including 100%. For numerical values it is to be understood that the terms “substantially”, “essentially”, “about”, and “approximately” may also relate to the range of 90%-110%, such as 95%-105%, especially 99%-101% of the values(s) it refers to.

The term “comprise” also includes embodiments wherein the term “comprises” means “consists of”.

The term “and/or” especially relates to one or more of the items mentioned before and after “and/or”. For instance, a phrase “item 1 and/or item 2” and similar phrases may relate to one or more of item 1 and item 2. The term “comprising” may in an embodiment refer to “consisting of” but may in another embodiment also refer to “containing at least the defined species and optionally one or more other species”.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The devices, apparatus, or systems may herein amongst others be described during operation. As will be clear to the person skilled in the art, the invention is not limited to methods of operation, or devices, apparatus, or systems in operation.

The term “further embodiment” and similar terms may refer to an embodiment comprising the features of the previously discussed embodiment, but may also refer to an alternative embodiment.

It should be noted that the above-mentioned embodiments illustrate rather than limit the invention, and that those skilled in the art will be able to design many alternative embodiments without departing from the scope of the appended claims.

In the claims, any reference signs placed between parentheses shall not be construed as limiting the claim.

Use of the verb “to comprise” and its conjugations does not exclude the presence of elements or steps other than those stated in a claim. Unless the context clearly requires otherwise, throughout the description and the claims, the words “comprise”, “comprising”, “include”, “including”, “contain”, “containing” and the like are to be construed in an inclusive sense as opposed to an exclusive or exhaustive sense; that is to say, in the sense of “including, but not limited to”.

The article “a” or “an” preceding an element does not exclude the presence of a plurality of such elements.

The invention may be implemented by means of hardware comprising several distinct elements, and by means of a suitably programmed computer. In a device claim, or an apparatus claim, or a system claim, enumerating several means, several of these means may be embodied by one and the same item of hardware. The mere fact that certain measures are recited in mutually different dependent claims does not indicate that a combination of these measures cannot be used to advantage.

The invention also provides a control system that may control the device, apparatus, or system, or that may execute the herein described method or process. Yet further, the invention also provides a computer program product, when running on a computer which is functionally coupled to or comprised by the device, apparatus, or system, controls one or more controllable elements of such device, apparatus, or system.

The invention further applies to a device, apparatus, or system comprising one or more of the characterizing features described in the description and/or shown in the attached drawings. The invention further pertains to a method or process comprising one or more of the characterizing features described in the description and/or shown in the attached drawings. Moreover, if a method or an embodiment of the method is described being executed in a device, apparatus, or system, it will be understood that the device, apparatus, or system is suitable for or configured for (executing) the method or the embodiment of the method, respectively.

The various aspects discussed in this patent can be combined in order to provide additional advantages. Further, the person skilled in the art will understand that embodiments can be combined, and that also more than two embodiments can be combined. Furthermore, some of the features can form the basis for one or more divisional applications.

1. A method for extraction of an organic acid (10) from a mixture (110), the method comprising:
an extraction stage comprising providing the mixture (110) to a sorbent (150);
an elution stage comprising passing an eluent (130) along the sorbent (120) to provide an eluate (140), wherein the eluent (130) comprises a solvent selected from the group consisting of acetone and alcohols, and wherein the eluent (130) comprises at least 0.1 M of OH⁻, and wherein the eluent (130) comprises at least 0.1 M of an cation selected from the group comprising Na⁺, K⁺, Ca²⁺, Mg²⁺ and NH₄⁺;
a precipitation stage comprising providing CO₂ (150) to the eluate (140), wherein CO₂ (150) and the cation form a carbonate, wherein the carbonate precipitates; and
a separation stage comprising separating the organic acid (10) from the eluate (140).

2. The method according to claim 1, wherein the organic acid (10) comprises an amino acid.

3. The method according to claim 1, wherein the organic acid (10) has a pKa≤4.0.

4. The method according to claim 1, wherein the sorbent (120) is selected from the group comprising a weak anion exchange resin, a strong anion exchange resin, and an adsorbent.

5. The method according to claim 1, wherein the solvent is selected from the group consisting of acetone, methanol, ethanol, and ethylene glycol, and wherein the eluent (130) comprises 30-90 wt. % of the solvent.

6. The method according to claim 1, wherein the eluent (130) comprises 10-50 wt. % water, and wherein the eluent (130) comprises at least 40 wt. % of the solvent.

7. The method according to claim 1, wherein the precipitation stage comprises providing the CO₂ (150) at a temperature selected from the range of 5-40° C.

8. The method according to claim 1, wherein the precipitation stage comprises separating the eluate (140) and the precipitated carbonate.

9. The method according to claim 1, wherein the cation comprises Na⁺.

10. The method according to claim 1, wherein the separation stage comprises providing CO₂ (150) to the eluate (140) to reduce the pH of the eluate (140) to a precipitation point of the organic acid (10).

11. The method according to claim 10, wherein the organic acid (10) comprises two or more organic acid compounds, and wherein the separation stage comprises providing CO₂ (150) to the eluate (140) to successively reduce the eluate to the precipitation points of the two or more organic acid compounds to separate the two or more organic acid compounds.

12. The method according to claim 1, wherein the mixture (110) is a waste stream.

13. A system (200) for recovery of an organic acid (10) from a mixture (110), the system (200) comprising an inlet (210), a sorbent container (220), an eluent supply (230), a separation unit (240), a carbon dioxide supply (250), wherein the eluent supply (230) is configured to provide an eluent (130) to the sorbent container (220), wherein the separation unit (240) is configured to receive an eluate (140) from the sorbent container (220), wherein the sorbent container (220) is configured to host a sorbent (120), and wherein the system (200) has an operational mode, wherein:
the inlet (210) is configured to provide the mixture (110) to the sorbent (120);
the eluent supply (230) is configured to provide the eluent (130) to the sorbent (120) to provide an eluate (140) to the separation unit (240), wherein the eluent (130) comprises 30-90 wt. % of a solvent selected from the group consisting of acetone and alcohols, and wherein the eluent (130) comprises at least 0.1 M of OH⁻, and wherein the eluent (130) comprises at least 0.1 M of an cation selected from the group comprising Na⁺, K⁺, Ca²⁺, Mg²⁺ and NH₄⁺;
the carbon dioxide supply (250) is configured to provide CO₂ (150) to the separation unit (240), wherein the CO₂ (150) and the cation form a carbonate, wherein the carbonate precipitates; and
the separation unit (240) is configured to separate the organic acid (10) from the eluate (140).

14. The system (200) according to claim 13, wherein the separation unit (240) comprises a first separation unit (241) and a second separation unit (242), wherein the first separation unit (241) is configured to receive an eluate (140) from the sorbent (120) wherein in the operational mode:
the carbon dioxide supply (250) is configured to provide CO₂ (150) to the first separation unit (241), wherein the first separation unit (241) is configured to separate the precipitated carbonate and remaining eluate (140), and to provide the eluate (140) to the second separation unit (242); and
the second separation unit (242) is configured to separate the organic acid (10) from the eluate (140).

15. The system (200) according to claim 14, wherein in the operational mode:
the carbon dioxide supply (250) is configured to provide CO₂ (150) to the second separation unit (242) to reduce the pH of the eluate (140) to a precipitation point of the organic acid (10).

16. (canceled)

17. A computer program product comprising instructions for execution on a control system (300) functionally coupled to a system (200), wherein the instructions, when executed by the control system (300), cause the system (200) to carry out the method according to claim 1.

18. A data carrier, carrying thereupon program instructions which, when executed by a control system (300) functionally coupled to a system (200), cause the system (200) to carry out the method according to claim 1.
